(19) United States
(12) Patent Application Publication
(10) Pub. No.: US 2013/0035739 A1
(43) Pub. Date: Feb. 7, 2013

(54) NERVE STIMULATION APPARATUS

(71) Applicant: Moe GOTO, Tokyo (JP)

(72) Inventor: Moe GOTO, Tokyo (JP)

(73) Assignee: OLYMPUS CORPORATION, Tokyo (JP)

(21) Appl. No.: 13/421,077

(22) Filed: Mar. 15, 2012

(30) Foreign Application Priority Data
Aug. 2, 2011 (JP) 2011-169474

(51) Int. Cl. A61N 1/365

(52) U.S. Cl. 607/25

(57) ABSTRACT

Nerve stimulation is performed while suppressing occurrence of symptom caused by sinus node dysfunction or atrioventricular node dysfunction. Provided is a nerve stimulation apparatus including: a stimulation signal generator configured to output a stimulation signal to a nerve; a cardiac-event acquisition unit configured to acquire cardiac events; and a control unit configured to control the stimulation signal generator such that sinus node dysfunction or atrioventricular node dysfunction is determined to be occurring and energy of the stimulation signal is reduced when a time interval value between the adjacent cardiac events acquired by the cardiac-event acquisition unit exceeds a predetermined threshold.

### CROSS-REFERENCE TO RELATED APPLICATIONS

This application is based on Japanese Patent Application No. 2011-169474, the contents of which are incorporated herein by reference.

### BACKGROUND OF THE INVENTION

1. Field of the Invention

The present invention relates to a nerve stimulation apparatus.

2. Description of Related Art

There is a conventionally known heart treatment device that detects changes in the heart's rhythm and adjusts a pulse intensity and a pulse duration for stimulating a nerve (for example, see Japanese Unexamined Patent Application, Publication No. 2006-280588).

### BRIEF SUMMARY OF THE INVENTION

The present invention provides a nerve stimulation apparatus capable of performing nerve stimulation while suppressing the occurrence of symptoms caused by sinus node dysfunction or atrioventricular node dysfunction.

The present invention provides the following solution.

According to one aspect, the present invention provides a nerve stimulation apparatus including: a stimulation signal generator configured to output a stimulation signal to a nerve; a cardiac-event acquisition unit configured to acquire cardiac events; and a control unit configured to control the stimulation signal generator such that sinus node dysfunction or atrioventricular node dysfunction is determined to be occurring and energy of the stimulation signal is reduced when a time interval value between the adjacent cardiac events acquired by the cardiac-event acquisition unit exceeds a predetermined threshold.

### BRIEF DESCRIPTION OF THE SEVERAL VIEWS OF THE DRAWINGS

FIG. 1 is a block diagram showing a nerve stimulation apparatus according to an embodiment of the present invention.

FIG. 2 is a flowchart for explaining a control method performed by a control unit of the nerve stimulation apparatus of FIG. 1.

FIG. 3 is a flowchart for explaining a detection process of sinus node dysfunction or atrioventricular node dysfunction in the flowchart in FIG. 2.

FIG. 4 is a histogram of RR intervals and frequencies thereof when sinus node dysfunction or atrioventricular node dysfunction is occurring.

FIG. 5 is a histogram of RR intervals and frequencies thereof when sinus node dysfunction or atrioventricular node dysfunction is not occurring.

FIG. 6 is a diagram showing a cumulative frequency curve of RR intervals when sinus node dysfunction or atrioventricular node dysfunction is occurring.

FIG. 7 is a flowchart showing a first modification of the detection process of FIG. 3.

FIG. 8 is a diagram showing (a) a waveform of an electrocardiac signal in the normal state and (b) a waveform of an electrocardiac signal when sinus node dysfunction or atrioventricular node dysfunction is occurring.

FIG. 9 is a flowchart showing a second modification of the detection process of FIG. 3.

FIG. 10 is a flowchart showing a third modification of the detection process of FIG. 3.

FIG. 11 is a flowchart showing a fourth modification of the detection process of FIG. 3.

### DETAILED DESCRIPTION OF THE INVENTION

A nerve stimulation apparatus 1 according to an embodiment of the present invention will be described below, with reference to the drawings.

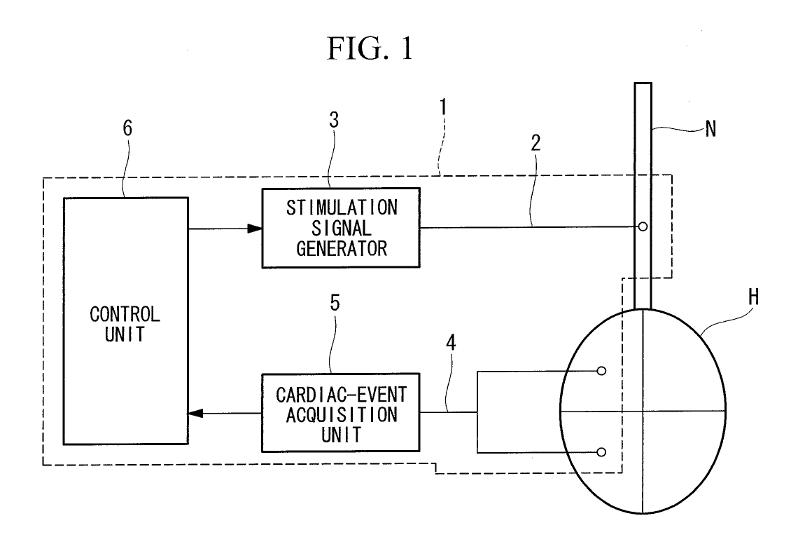

As shown in FIG. 1, the nerve stimulation apparatus 1 according to this embodiment is provided with a stimulation signal generator 3 connected to a vagus nerve N, which is connected to a heart H, through a nerve stimulation lead 2, a cardiac-event acquisition unit 5 connected to the heart H through a detection lead 4, and a control unit 6 that controls the stimulation signal generator 3 on the basis of a cardiac event acquired by the cardiac-event acquisition unit 5.

The nerve stimulation lead 2 has two electrodes (not shown) at its tip and stimulates the vagus nerve (nerve) N by applying a voltage across these electrodes.

The stimulation signal generator 3 generates a stimulation pulse train for electrically stimulating the vagus nerve N on the basis of an instruction signal from the control unit 6 and supplies the generated stimulation pulse train to the vagus nerve N via the nerve stimulation lead 2.

The detection lead 4 has an electrode (not shown) at its tip, and a plurality of detection leads 4 are placed so as to have each electrode be in contact with the respective parts of the heart H. For example, the detection leads 4 are connected to the right atrium, the right ventricle, and the left ventricle.

The cardiac-event acquisition unit 5 detects a voltage change independently obtained with each tip electrode of the detection leads 4. As the cardiac event, for example, a P wave, an R wave, and a T wave of an electrocardiac signal, or all events acquired from the electrocardiac signal can be utilized. In this description, for example, an R wave of the electrocardiac signal is utilized as the cardiac event.

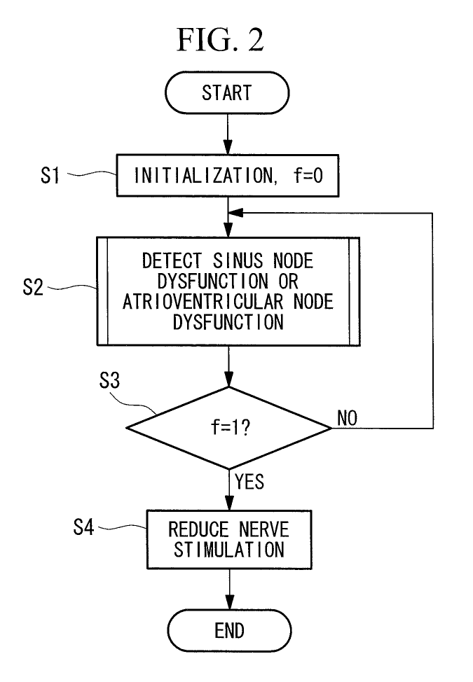

As shown in FIG. 2, while performing the vagus nerve stimulation, the control unit 6 first initializes a flag (f=0) indicating sinus node dysfunction or atrioventricular node dysfunction (Step S1) and performs a detection process for detecting whether or not sinus node dysfunction or atrioventricular node dysfunction is occurring based on the cardiac event detected by the detection lead 4 (Step S2). As a result of the detection process, it is determined whether or not the occurrence of sinus node dysfunction or atrioventricular node dysfunction has been detected (Step S3). If the occurrence of sinus node dysfunction or atrioventricular node dysfunction has been detected (f=1), the stimulation signal generator 3 is controlled so that the stimulation energy of the nerve stimulation is reduced (Step S4). Here, the reduction of the stimulation energy includes reducing the pulse voltage, shortening the pulse duration, extending the pulse interval, and so forth.

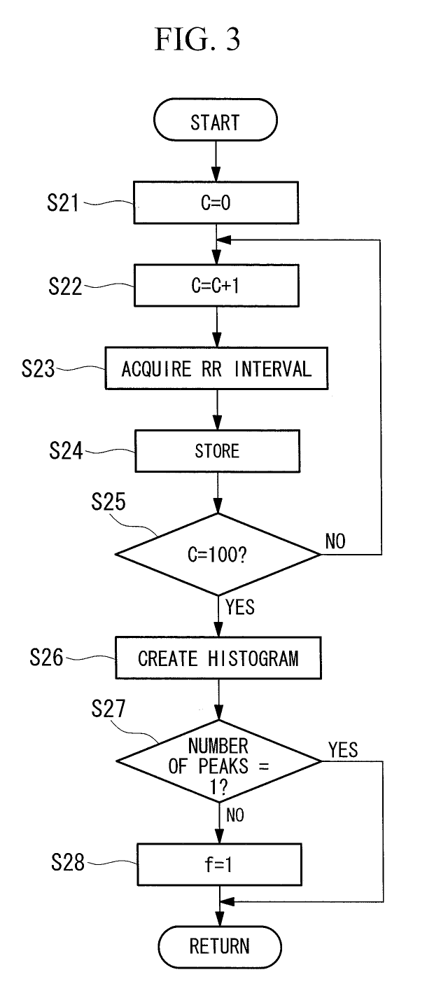

As shown in FIG. 3, a counter C is first reset in the detection process of sinus node dysfunction or atrioventricular node dysfunction (Step S21). A step of incrementing the counter C (Step S22), a step of obtaining from the electrocardiac signal the value of a time interval (RR interval) between peaks of each R wave (Step S23), and a step of storing the value (Step S24) are then repeated until the counter C counts 100 (Step S25). The values of the RR interval for 100 beats are thereby obtained. The number of beats is not limited to 100 beats, and the values may be obtained for any number of beats other than 100 beats.

Next, a histogram, in which RR interval is taken as the bin and the frequency is taken as the frequency distribution, is generated by using the RR intervals stored for 100 beats (Step S26).

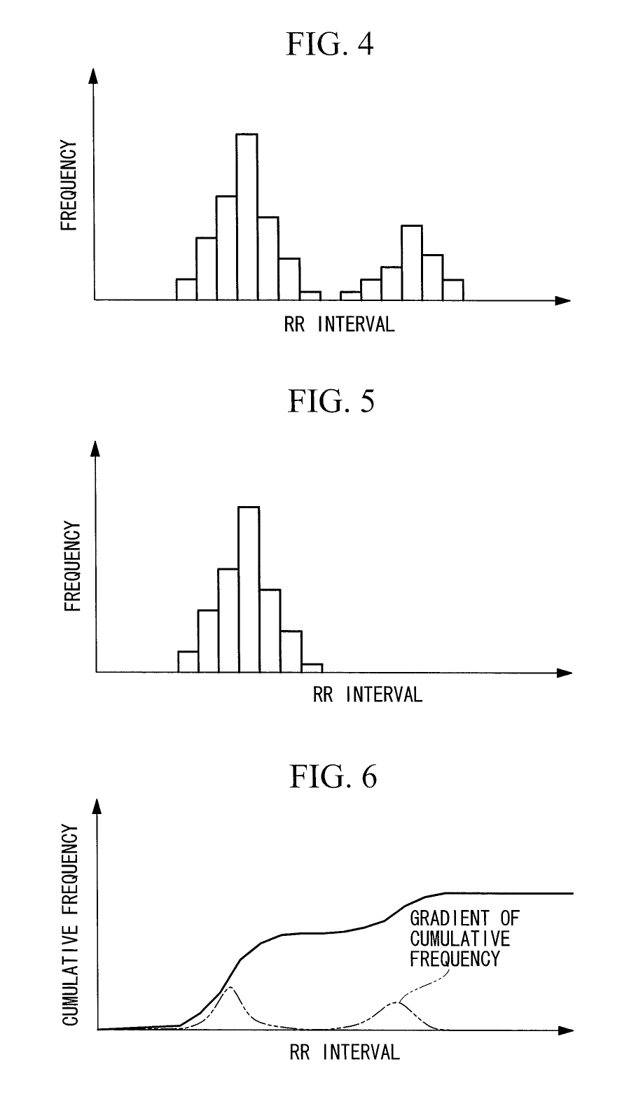

It is then determined whether or not there is more than one bin of the time intervals forming peaks by analyzing the distribution in the generated histogram (Step S27). As shown in FIG. 4, if there is more than one bin of the time intervals forming peaks, a flag f indicating sinus node dysfunction or atrioventricular node dysfunction is set to f=1 (Step S28), and the process proceeds to a determination step S3. On the other hand, as shown in FIG. 5, if there is a single peak, f=0 is maintained, and the process proceeds to the determination step S3.

As described above, according to the nerve stimulation apparatus 1 of this embodiment, it is possible to prevent the vagus nerve N from being continuously stimulated with high energy when sinus node dysfunction or atrioventricular node dysfunction is occurring. By doing so, an advantage is afforded in that it is possible to prevent the occurrence of an inconvenience causing discomfort, dizziness, fainting, and so forth due to sinus node dysfunction or atrioventricular node dysfunction. In addition, by generating the histogram, it is possible to analyze the data statistically and to detect sinus node dysfunction or atrioventricular node dysfunction more accurately.

In this embodiment, the histogram is generated from the cumulated RR intervals for 100 beats in a detection process of sinus node dysfunction or atrioventricular node dysfunction; however, instead of generating the histogram, a cumulative frequency curve, such as the solid line shown in FIG. 6, may be generated, and sinus node dysfunction or atrioventricular node dysfunction may be detected as occurring if there is more than one peak when the cumulative frequency curve is differentiated (shown with a broken line in FIG. 6).

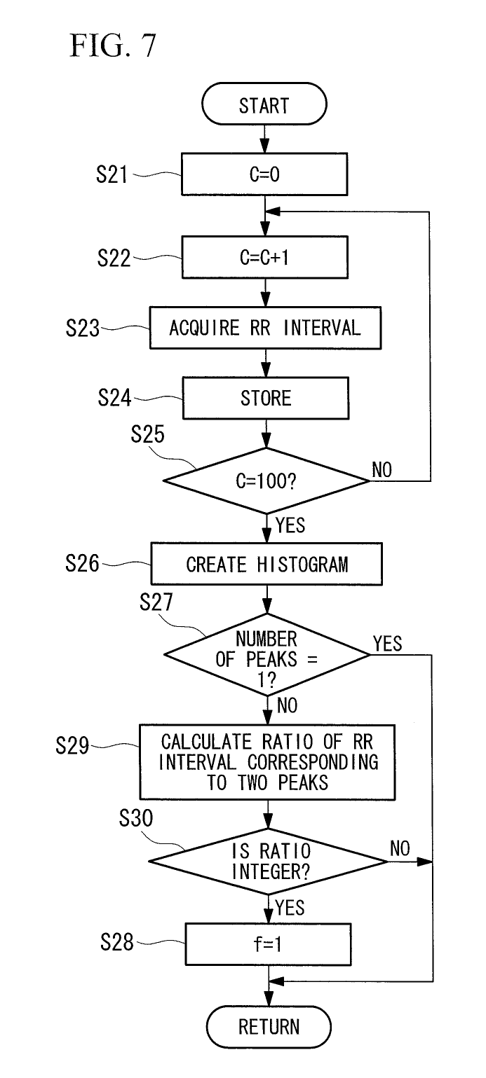

In addition, when there is more than one peak in the histogram or more than one peak is formed by gradient of the accumulative frequency curve, sinus node dysfunction or atrioventricular node dysfunction is determined to be occurring, and the flag f is set to f=1; instead of this, however, as shown in FIG. 7, sinus node dysfunction or atrioventricular node dysfunction may be determined to be occurring and the flag f may be set to f=1 when more than one value of the RR intervals forming peaks is extracted (Step S29) and there is a relationship such that the larger value is an integer multiple of two or more times the smaller value (Step S30).

In this case, the relationship that A is an integer multiple of two or more times B means that, in addition to a strict integer multiple relationship, the relationship satisfies the following relational expression.

B×n×0.8≦A≦B×n×1.2

In the expression, n is an integer of 2 or more.

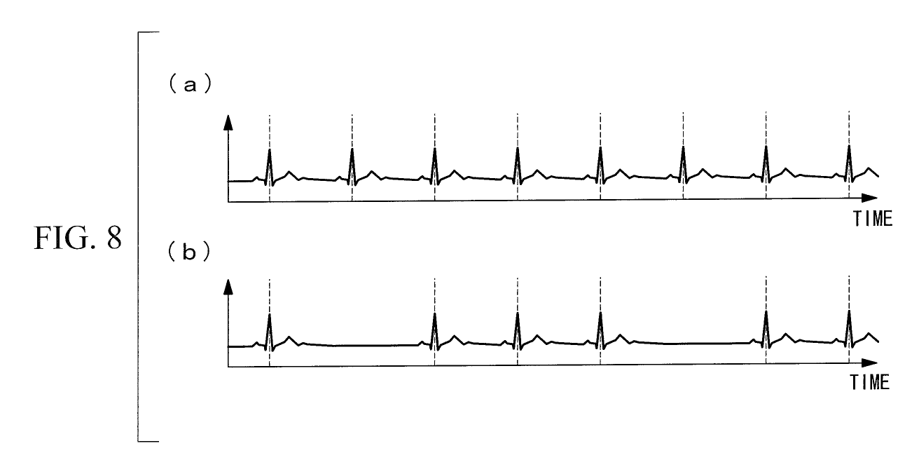

When sinus node dysfunction or atrioventricular node dysfunction occurs, because there are some missing beats in the heartbeat, as shown in (b) in FIG. 8, in contrast to the normal periodical heartbeat shown in (a) in FIG. 8, an approximately integer multiple relationship is found if their RR intervals are compared; therefore, by detecting this relationship, it is possible to detect the occurrence of sinus node dysfunction or atrioventricular node dysfunction more accurately.

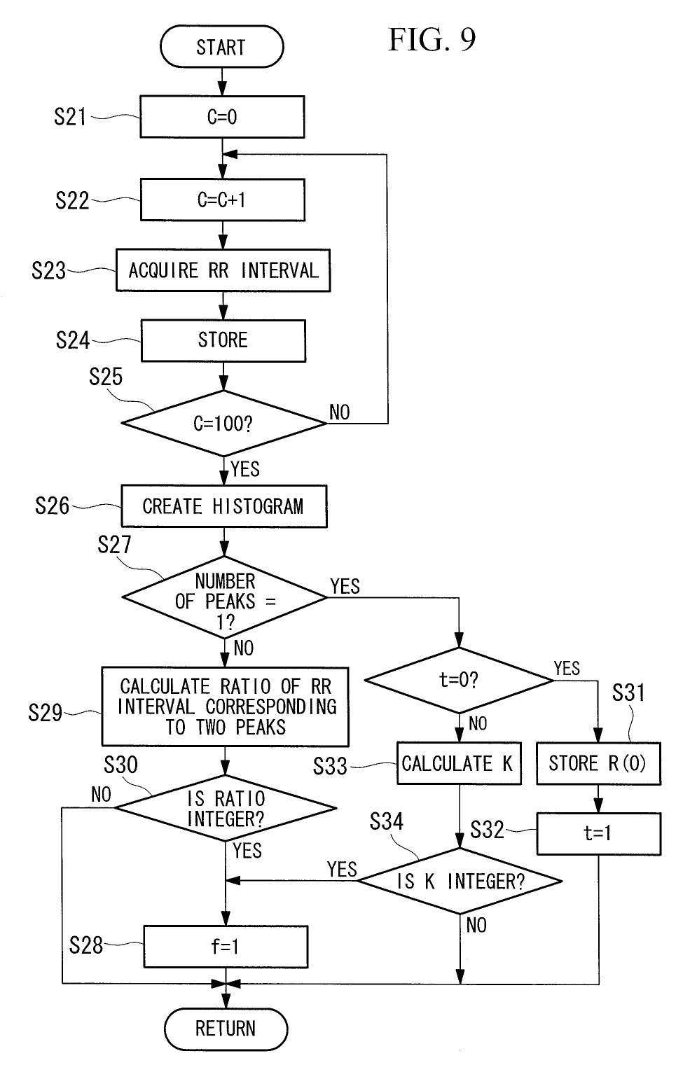

In addition, in this embodiment, sinus node dysfunction or atrioventricular node dysfunction is detected to be occurring only if there is more than one bin of the time intervals forming peaks in the histogram; instead of this, however, sinus node dysfunction or atrioventricular node dysfunction may be detected by following the detection process shown in FIG. 9 even in the case where a single peak is present in the histogram due to an insufficient heart rate on all cardiac events that have caused extension of the time intervals of all peaks.

In other words, a flag t is also initialized (t=0) in the initializing step S1, and determination of the cardiac event is performed in the normal state in which sinus node dysfunction or atrioventricular node dysfunction is not occurring. At this time, if a single peak is found in the histogram generated during the initial detection process, a bin value R(0) of the RR intervals forming that peak is stored in a storage unit (not shown) (Step S31), and the flag t is set to t=1 (Step S32).

In the case where the histogram generated thereafter shows a single peak, a ratio K=R(t)/R(0) of a bin value R(t) of the RR intervals forming that peak to the bin value R(0) of the initial RR intervals is calculated (Step S33), it is determined whether or not the ratio K calculated is an integer of equal to 2 or more (Step S34), sinus node dysfunction or atrioventricular node dysfunction is also detected to be occurring when the ratio K is an integer of equal to 2 or more, and the flag f is set to f=1.

By doing so, an advantage is afforded in that it is possible to detect the occurrence of sinus node dysfunction or atrioventricular node dysfunction and to prevent, with higher reliability, the occurrence of an inconvenience causing discomfort, dizziness, fainting, and so forth due to sinus node dysfunction or atrioventricular node dysfunction even in the case where more than one peak is not present in the histogram and the ratio K of the bin value of the RR intervals forming a single peak in the generated histogram to the bin value of the RR intervals forming a single peak in a histogram in the normal state is an integer of equal to 2 or more.

When there is a single peak in the initially generated histogram, the bin value R(0) of the RR intervals forming the peak is stored in a storage unit (not shown), and the ratio K is calculated therewith; instead of this, however, the RR interval value in the normal state may be stored in a storage unit, and the ratio may be calculated with the stored RR interval value in the normal state.

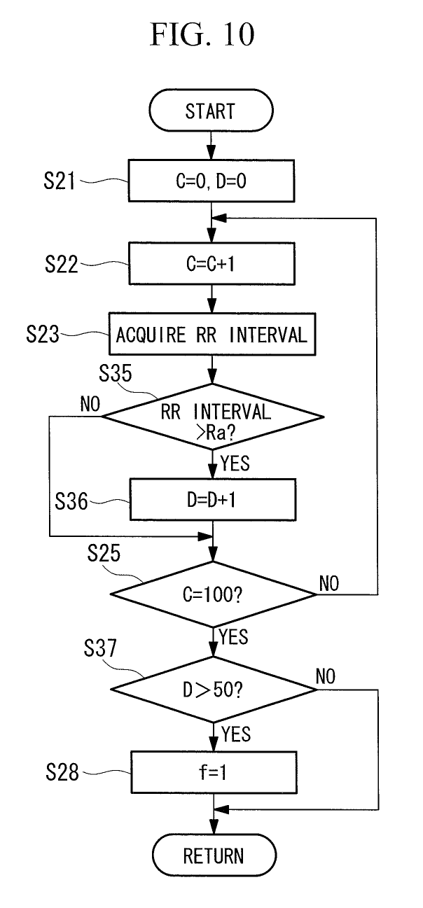

In addition, in each of the above-described embodiments, although a histogram or a cumulative frequency curve is generated, it is not limited thereto. For example, as shown in FIG. 10, RR interval values for 100 beats may be successively stored in a storage unit (not shown), each of the RR interval values may be successively compared with a default value Ra (for example, a value 1.5 times the RR interval value in the normal state) for every beat (Step S35), the number of the RR intervals that are greater than the default value Ra may be integrated (Step S36), and sinus node dysfunction or atrioventricular node dysfunction may be determined to be occurring when the integrated value is equal to or greater than the predetermined threshold (for example, 50) (Step S37).

In addition, in the case where a ratio is calculated for every beat with the RR interval value of a previous beat, sinus node dysfunction or atrioventricular node dysfunction may be determined to be occurring if the ratio is an integer of equal to 2 or more (or 1/(an integer equal to 2 or more)).

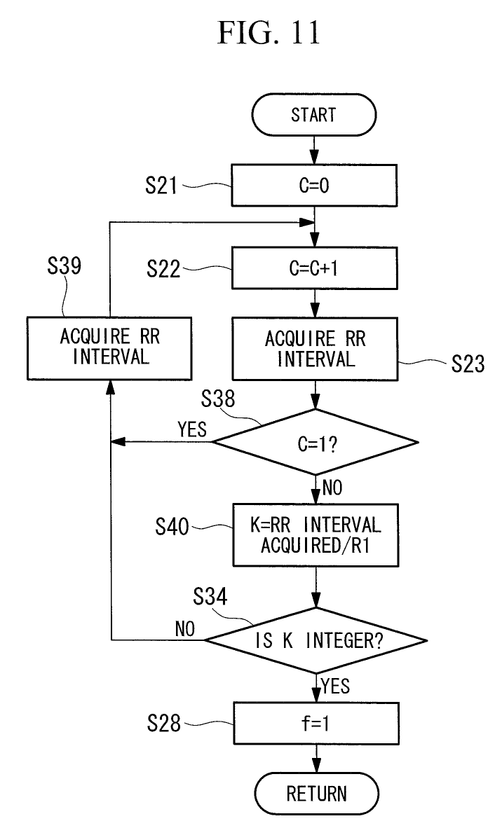

For example, as shown in FIG. 11, the initially acquired RR interval value is stored in a storage unit (not shown) as R1 (Step S38, S39), the ratio K is calculated by dividing the RR interval value acquired thereafter by the stored R1 (Step S40), and when the ratio K is an integer equal to 2 or more, sinus node dysfunction or atrioventricular node dysfunction may be determined to be occurring. If the ratio K is not an integer, the process updates R1 with the RR intervals acquired (Step S39).

According to such a method in which a histogram or a cumulative frequency curve is not generated, an advantage is afforded in that it is possible to detect sinus node dysfunction or atrioventricular node dysfunction in a short period of time.

In this embodiment, although an R wave in the electrocardiac signal is illustrated as a cardiac event, it is not limited thereto; other cardiac events such as a P wave, a T wave, and so forth may be employed. In addition, determination of sinus node dysfunction or atrioventricular node dysfunction may commence by detecting that there is no body motion for a certain period of time, a cardiac event interval is extended, or time is in a predetermined time period, and by using such detection as a trigger. By performing determination of sinus node dysfunction or atrioventricular node dysfunction when a predetermined condition is satisfied, an advantage is afforded in that it is possible to eliminate wasteful processing required for generating a histogram.

What is claimed is:

1. A nerve stimulation apparatus comprising:
a stimulation signal generator configured to output a stimulation signal to a nerve;
a cardiac-event acquisition unit configured to acquire cardiac events; and
a control unit configured to control the stimulation signal generator such that sinus node dysfunction or atrioventricular node dysfunction is determined to be occurring and energy of the stimulation signal is reduced when a time interval value between the adjacent cardiac events acquired by the cardiac-event acquisition unit exceeds a predetermined threshold.

2. A nerve stimulation apparatus according to claim 1, wherein the control unit generates a histogram from a plurality of cardiac events acquired by the cardiac-event acquisition unit by taking the time interval as a bin and determines whether or not sinus node dysfunction or atrioventricular node dysfunction is occurring on the basis of the histogram.

3. A nerve stimulation apparatus according to claim 2, wherein the control unit determines that sinus node dysfunction or atrioventricular node dysfunction is occurring when there is more than one bin of the time interval forming peaks in a frequency distribution of the histogram generated by the control unit.

4. A nerve stimulation apparatus according to claim 3, wherein the control unit compares bin values of the time intervals forming peaks in the frequency distribution of the histogram generated by the control unit and determines that sinus node dysfunction or atrioventricular node dysfunction is occurring when there is a relationship such that the bin value of one time interval is an integer multiple of two or more times the bin value of the other time interval.

5. A nerve stimulation apparatus according to claim 2, further comprising a storage unit in which a histogram in a normal state is stored, wherein
the control unit compares a bin value of the time interval forming a peak in a frequency distribution of the normal-state histogram stored in the storage unit and a bin value of the time interval forming a peak in a frequency distribution of the histogram generated by the control unit and determines that sinus node dysfunction or atrioventricular node dysfunction is occurring when there is a relationship such that the bin value of the time interval forming a peak in the frequency distribution of the histogram generated by the control unit is an integer multiple of two or more times the bin value of the time interval forming a peak in the frequency distribution of the normal-state histogram stored in the storage unit.

6. A nerve stimulation apparatus according to claim 2, further comprising a storage unit in which a time interval value of a cardiac event in a normal state is stored, wherein
the control unit determines that sinus node dysfunction or atrioventricular node dysfunction is occurring when there is a relationship such that the bin values of the time interval forming peaks in a frequency distribution of the currently generated histogram is an integer multiple of two or more times the stored time interval value in normal state.

7. A nerve stimulation apparatus according to claim 1, further comprising a storage unit in which time intervals between the adjacent cardiac events acquired by the cardiac-event acquisition unit are successively stored, wherein
the control unit compares a time interval value newly acquired and a time interval value stored and determines that sinus node dysfunction or atrioventricular node dysfunction is occurring when there is a relationship such that one time interval value is an integer multiple of two or more times the other time interval value.
